(19) United States
(12) Plant Patent Application Publication
(10) Pub. No.: US 2004/0221351 P1
(43) Pub. Date: Nov. 4, 2004

(54) Aster plant named "Diamond"

(76) Inventors: Harry W.M. van Straalen, Rijsenhout (NL); Wilfried J. Poland, Wervershoof (NL)

Correspondence Address: PARKHURST & WENDEL, SUITE 210, 1421 PRINCE STREET, ALEXANDRIA, VA, 22314, US

(21) Appl. No.: 10/426,847

(22) Filed: May 1, 2003

(51) Int. Cl. A01H 5/00

(52) U.S. Cl. PLT/355

(57) ABSTRACT

An Aster plant named ‘Diamaster’ characterized by its small sized blooms with white ray-florets and yellow disc florets, which can be propagated by means of cuttings from cuttings and produced within a short period.

### RELATED CULTIVARS

‘Diamond’ is related to ‘Dynasty’ (copending USPP application) and ‘Duke’ (copending USPP application), which are all aster varieties developed from similar breeding programs.

### BACKGROUND OF THE INVENTION

‘Diamond’ is a product of a breeding-program which had the objective of creating new Aster cultivars, which can be grown as pot plants and propagated by means of cuttings from cuttings, similar to the cultivation and propagation of all year round pot chrysanthemum. The new plant of the present invention comprises a new and distinct cultivar of an Aster plant. ‘Diamond’ is a seedling from a cross in a breeding program maintained under the control of inventor. The female parent is an unnamed seedling not available to inventor for description. The male parent is unknown, being a mixed population of a group of male parents. The new and distinct cultivar was discovered and selected as a flowering plant within the progeny of the stated cross by Harry W. M. van Stralen in a controlled environment (greenhouse) in Rijsenhout Holland in 1996. The first act of asexual reproduction of ‘Diamond’ was accomplished when vegetative cuttings were taken from the initial selection in 1997 in a controlled environment in Rijsenhout Holland.

### SUMMARY OF THE INVENTION

The present invention is a new and distinct variety of Aster bearing medium sized blooms with white ray-florets and yellow disc florets, which can be propagated by cuttings from a cuttings and produced as pot asters in 8 weeks time.

### BRIEF DESCRIPTION OF THE DRAWINGS

The present invention of a new and distinct variety of Aster is shown in the accompanying drawings, the color being as nearly true as possible with color photographs of this type.

FIG. 1 shows a plant of the cultivar in full bloom.

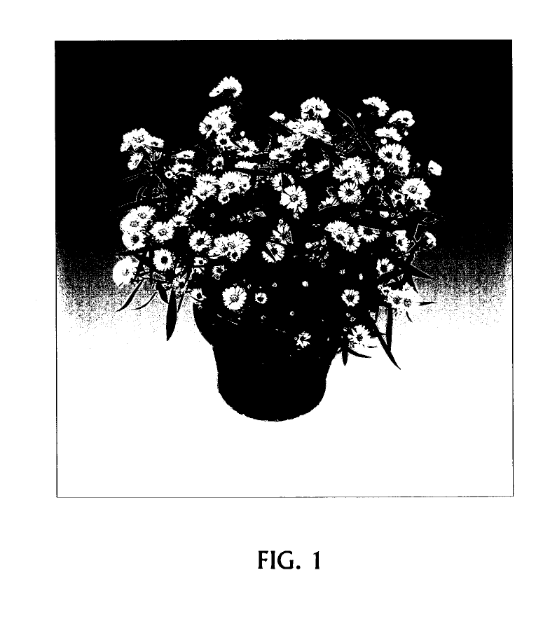

FIG. 2 shows the various stages of bloom of the new cultivar.

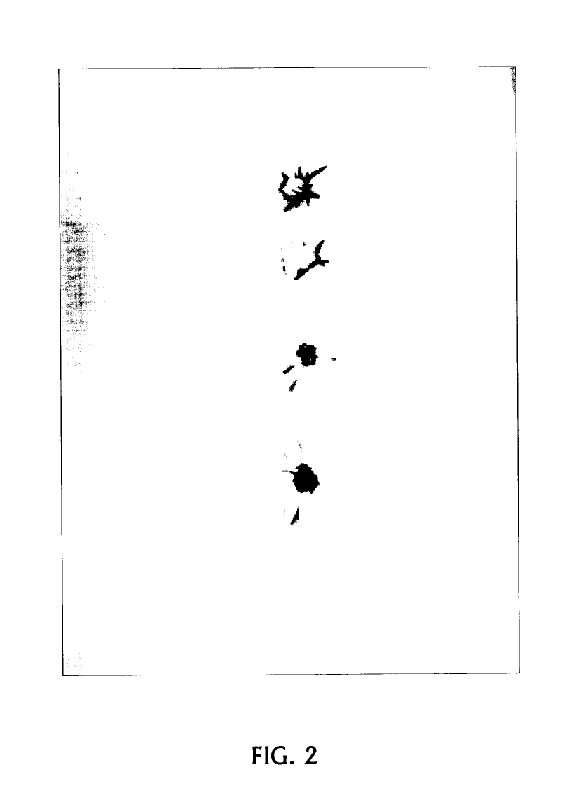

FIG. 3 shows the foliage of the new cultivar

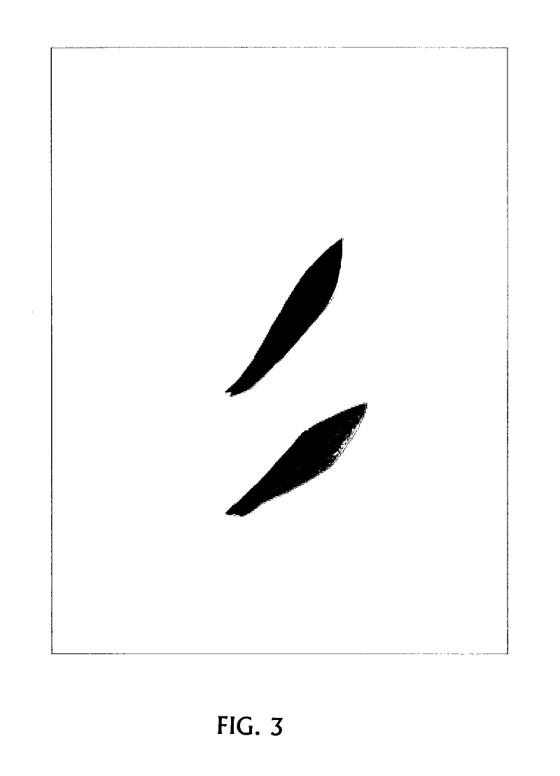

### DESCRIPTION OF THE INVENTION

This new variety of Aster is of the botanical classification Aster novi-belgii L. The observations and measurements were gathered from plants grown in a greenhouse in Rijsenhout Holland in a photo-periodic controlled crop under conditions generally used in commercial practice. The greenhouse temperatures during this crop were at day-time between 18° C. and 25° C. and at night 20° C. The photo-periodic response time in this crop was 42 days after an average of 16 long days after sticking of the unrooted cuttings. Plants are pinched ten days after sticking. Growth retardants were applied in an average dose of 1.5 gram/liter water, starting one week after pinching. The plants were observed (directly) during the flowering of this crop. The plant is susceptible to Powdery Mildew. No tests were done on cold or drought tolerance. This new variety produces medium sized blooms with white ray-florets and yellow disc-florets blooming on the plant for 4 weeks. This new variety of Aster has been found to retain its distinctive characteristics throughout successive propagations, however the phenotype may vary significantly with variations in environment such as light intensity and temperature. ‘Diamond’ can be planted with assimilation lightning (high pressure sodium lamps) between week 5 and week 35 under greenhouse conditions in Holland.

From the cultivars known to inventor the most similar existing cultivar in comparison to ‘Daimond’ is ‘Margrethe Viking’ (U.S. Plant Pat. No. 10,358). When ‘Margrethe Viking’ and ‘Diamond’ are being compared the following differences are noticed: The differences of ‘Margrethe Viking’ and ‘Diamond’ are (1) Method of propagation. (2) Production time (3) Size of bloom. (1) In the propagation of ‘Margrethe Viking’, motherplants for the production of cuttings originate from in-vitro material, while in the case of ‘Diamond’ motherplants can be directly obtained as cuttings from cuttings. (2) The total time needed to obtain flowering time is different, for ‘Margrethe Viking’ this is 11 weeks while for ‘Dynasty’ this amounts to 8 weeks. (3) The size of the bloom of ‘Margrethe Viking ’ is larger than that of ‘Diamond’.

The following is a description of the plant and characteristics that distinguish ‘Daimond’ as a new and distinct variety.

The color designations are taken from the plant itself. Accordingly, any discrepancies between the color designations and the colors depicted in the photographs are due to photographic tolerances. The color chart used in this description is: The Royal Horticultural Society Colour Chart, edition 1995.

Bud:

Size.—Small; cross-section 0.2 cm, height 0.4 cm.

Outside color.—Yellow-green 154 D.

Involucral bracts.—2 rows, length 4 mm, width 1 mm .

Involucral bracts among disc-florets.—Not present.

Involucral bracts color.—Green 138 D.

Bloom:

Type.—Daisy.

Size.—Small.

Fully expanded.—2.4 cm.

Number of blooms per branch.—22.

Peduncle length.—3 cm.

Peduncle color.—Green 139 B.

Peduncle angle.—30°.

Performance on the plant.—4 weeks.

Seeds.—Produced in small quantities, oval/ovate shaped, grey-brown 199 A, 1 mm. in length.

Color:

Center of the flower (disc-florets).—Immature Yellow-green 151 A Mature Green-yellow 1 A.

Color of the ray-florets.—Upper surface: White 155 D Lower surface: White 155 D.

Tonality from distance.—A pot aster with white flowers and a yellow disc.

Color of the surface of the ray-florets after aging of the plant.—White 155 D.

Ray florets:

Number of whorls of ray-florets.—2.

Texture.—Upper and under side smooth.

Number of ray-florets.—25.

Shape in cross-section.—Straight.

Curvature of longitudinal axis.—Flat.

Length of corolla tube.—0.4 cm.

Ray-floret length.—1.7 cm.

Ray-floret width.—0.3 cm.

Shape of tip.—Rounded.

Shape of apex.—Acute.

Disc florets:

Disc diameter.—0.5 cm.

Distribution of disc-florets.—Numerous, clearly visible at all stages of flowering.

Shape.—Funnel.

Lenght.—0.5 cm.

Color.—Yellow-green 145 D.

Reproductive organs:

Stamen.—Present in disc florets only.

Stamen color.—Yellow 13 A.

Pollen.—Present.

Pollen color.—Yellow-orange 14 A.

Styles.—Present in both Ray and Disc-florets.

Style color.—Yellow-green 150 D.

Style length.—0.6 cm.

Ovaries.—Enclosed in calyx.

Calyx shape.—Pappus.

Calyx lenght.—0.4 cm.

Calyx color.—Yellow-green 150 D.

Plant:

Form.—A pot aster meant for indoor use.

Growth habit.—Upright growing and outwardly spreading.

Growth rate.—Vigorous.

Height.—22 cm.

Width.—10 cm.

Internode lenght.—3 cm.

Stem diameter.—1.5-2 mm.

Stem color.—Green 139 B.

Stem strength.—Strong.

Stem brittleness.—Not brittle.

Stem anthocyanin coloration.—Absent.

Length of lateral branch.—From top to bottom 16 cm.

Lateral branch color.—Green 139 B.

Lateral branch, attachment.—25°.

Branching (average number of lateral branches).—Good with 3 breaks after pinching.

Flowering response (photo-periodic controlled crop, not natural season).—42 Days.

Foliage:

Leaf arrangement.—Alternate.

Color immature stage.—Upper side Yellow-green 146 C Under side Yellow-green 147 B.

Color mature stage.—Upper side Green 137 B Under side Green 137 C.

Color midvein.—Upper side Yellow-green 147 D Under side Yellow-green 147 D.

Size.—Small; length 7.5 cm, width 1.2 cm.

Quantity (number per lateral branch).—14-15.

Shape.—Oblanceolate.

Texture upper side.—Glabrous.

Texture under side.—Glabrous.

Venation arrangement.—Pinnately netted.

Shape of the margin.—Sinuate.

Shape of base.—Attenuate.

Apex.—Acuminate.

Differences with the comparison varieties

| | ‘Diamond’ | ‘Margrethe Viking’ |
|---|---|---|
| Method of Propagation | Cuttings from cuttings | In-vitro cycle |
| | | necessary |
| Production time | 8 weeks | 11 weeks |
| Bloom size | Small | Large |

I claim:

1. A new and distinct variety of Aster plant as described and illustrated.
